SEQUENCE LISTING (17 sequences)

SEQ ID NO: 1 - DNA, 22 bp, Artificial Sequence
  Description of Artificial Sequence Synthetic oligonucleotide
  Lpn FOR primer
ttgtcttatagcattggtgccg

SEQ ID NO: 2 - DNA, 21 bp, Artificial Sequence
  Description of Artificial Sequence Synthetic oligonucleotide
  Lpn REV primer
ccaattgagcgccactcatag

SEQ ID NO: 3 - DNA, 20 bp, Artificial Sequence
  Description of Artificial Sequence Synthetic oligonucleotide
  Lpn Probe
  misc_feature (1)...(1): Cal-610 labelled
  misc_feature (20)...(20): BHQ-2 Labelled
caattgagcgccactcatag

SEQ ID NO: 4 - DNA, 8 bp, Artificial Sequence
  Description of Artificial Sequence Synthetic oligonucleotide
  S3_WT_MBP-1
atccaggg

SEQ ID NO: 5 - DNA, 31 bp, Artificial Sequence
  Description of Artificial Sequence Synthetic oligonucleotide
  S3_WT_probe
  misc_feature (1)...(1): FAM labelled
  misc_feature (31)...(31): BHQ-1 labelled
ccgacgtgtaagcaccccctggatccgtcgg

SEQ ID NO: 6 - DNA, 22 bp, Artificial Sequence
  Description of Artificial Sequence Synthetic oligonucleotide
  Legionella forward primer
ttgtcttatagcattggtgccg

SEQ ID NO: 7 - DNA, 21 bp, Artificial Sequence
  Description of Artificial Sequence Synthetic oligonucleotide
  Legionella reverse primer
ccaattgagcgccactcatag

SEQ ID NO: 8 - DNA, 25 bp, Artificial Sequence
  Description of Artificial Sequence Synthetic oligonucleotide
  Legionella probe
  misc_feature (1)...(1): CalFluor610 labelled
  misc_feature (25)...(25): BHQ-2 labelled
cggaagcaatggctaaaggcatgca

SEQ ID NO: 9 - DNA, 23 bp, Artificial Sequence
  Description of Artificial Sequence Synthetic oligonucleotide
  RNaseP probe
  misc_feature (1)...(1): FAM labelled
  misc_feature (23)...(23): BHQ-1 labelled
ttctgacctgaaggctctgcgcg

SEQ ID NO: 10 - DNA, 113 bp, Artificial Sequence
  Description of Artificial Sequence Synthetic polynucleotide
  IAC recombinant plasmid insert
ttgtcttatagcattggtgccgatttggggaagaattttaaaaatcaaggcatagatgttaatcttctgacctgaaggctctgcgcgagacgctatgagtggcgctcaattgg

SEQ ID NO: 11 - DNA, 22 bp, Artificial Sequence
  Description of Artificial Sequence Synthetic oligonucleotide
  Legionella forward primer
ttgtcttatagcattggtgccg

SEQ ID NO: 12 - DNA, 21 bp, Artificial Sequence
  Description of Artificial Sequence Synthetic oligonucleotide
  Legionella reverse primer
ccaattgagcgccactcatag

SEQ ID NO: 13 - DNA, 25 bp, Artificial Sequence
  Description of Artificial Sequence Synthetic oligonucleotide
  Legionella probe
  misc_feature (1)...(1): CalFluor610 labelled
  misc_feature (25)...(25): BHQ-2 labelled
cggaagcaatggctaaaggcatgca

SEQ ID NO: 14 - DNA, 20 bp, Artificial Sequence
  Description of Artificial Sequence Synthetic oligonucleotide
  RnaseP forward primer
ggacggtcatgggacttcag

SEQ ID NO: 15 - DNA, 19 bp, Artificial Sequence
  Description of Artificial Sequence Synthetic oligonucleotide
  RnaseP reverse primer
aaggtgagcggctgtctcc

SEQ ID NO: 16 - DNA, 23 bp, Artificial Sequence
  Description of Artificial Sequence Synthetic oligonucleotide
  RnaseP probe
  misc_feature (1)...(1): FAM labelled
  misc_feature (23)...(23): BHQ-1 labelled
ttctgacctgaaggctctgcgcg

SEQ ID NO: 17 - DNA, 115 bp, Artificial Sequence
  Description of Artificial Sequence Synthetic polynucleotide
  Non-competitive IAC oligonucleotide
ggacggtcatgggacttcagcatggcggtgtttgcagatttggacctgcgagcgggatctatcacattctgacctgaaggctctgcgcggacttgtggagacagccgctcacctt

(19) United States
(12) Patent Application Publication
(10) Pub. No.: US 2022/0282302 A1
(43) Pub. Date: Sep. 8, 2022

(54) LINEARLY-AMPLIFIED INTERNAL CONTROL FOR NUCLEIC ACID AMPLIFICATION REACTION

(71) Applicant: Genomadix Inc., Ottawa (CA)

(72) Inventors: Chris Harder, Dunrobin (CA); Jeffrey Do, Ottawa (CA); Andy Lee, Ottawa (CA)

(21) Appl. No.: 17/697,481

(22) Filed: Mar. 17, 2022

Related U.S. Application Data
(62) Division of application No. 16/981,134, filed on Sep. 15, 2020.
(60) Provisional application No. 62/643,897, filed on Mar. 16, 2018.

(51) Int. Cl. C12Q 1/686; C12Q 1/6848; C12Q 1/6876

(52) U.S. Cl. CPC C12Q 1/686; C12Q 1/6848; C12Q 1/6876; C12Q 2521/101; C12Q 2527/143; C12Q 2527/149; C12Q 2531/101; C12Q 2533/101; C12Q 2545/101

(57) ABSTRACT

The present disclosure provides, among other things, systems, methods, and kits include internal amplification controls. Provided internal amplification controls are or include non-target sequences that are amplified during nucleic acid amplification. Provided internal amplification controls are linearly amplified. Provided internal amplification controls are useful for systems, methods and kits for nucleic acid amplification.

### RELATED APPLICATIONS

The present application claims the benefit of U.S. Provisional Patent Application Ser. No. 62/643,897, filed on Mar. 16, 2018, titled “Linearly-Amplified Internal Control,” the disclosure of which is incorporated herein by reference in its entirety.

### BACKGROUND

Nucleic acid amplification technology is used to amplify nucleic acids or specific regions of nucleic acids. The amplification process is capable of taking extremely small amounts of a nucleic acid sample and generating copies of a particular sequence, portion or fragment thereof. An internal amplification control (IAC) includes a non-target sequence present during the amplification process used to reveal amplification failures.

### SUMMARY

Among other things, the present disclosure provides systems useful for amplification of nucleic acids. The present disclosure also provides methods of using such amplification systems. Implementations of the present disclosure are useful with a wide range of applications, including but not limited to: cloning, disease detection, disease diagnosis, environmental testing, forensic analysis, genetic mapping, genetic testing, nucleic acid sequencing, tissue typing, etc.

The present disclosure encompasses a recognition that nucleic acid amplification reactions (e.g., PCR) are sensitive to inhibition, e.g., due to inhibitory substances present in a nucleic acid sample. An internal amplification control (IAC), e.g., an oligonucleotide with a known sequence, can be used to assess various types of inhibition. An IAC may be a competitive (e.g., the same primers can bind to both sample and IAC) or non-competitive (e.g., different primers bind to sample and IAC, respectively). In general for PCR reactions, both sample and the IAC may be present in a reaction mixture at known quantities/concentrations, and may be amplified exponentially using standard PCR techniques. A limitation of such IACs is that they can interfere with detection or quantification of an unknown sample. Thus, the outcome of reactions in the presence of IAC may be highly sensitive to the relative quantities of sample and IAC present in a reaction mixture. The present disclosure encompasses a recognition that an internal amplification control that is linearly amplified consumes nucleic acid reagents at a lower rate than that of an exponentially (or linearly) amplified target sample. An example IAC may be amplified using a single (specific) primer binding to the IAC, resulting in one amplicon per cycle instead of two. A linearly amplified IAC may provide a more robust amplification reaction whose results are less sensitive to relative concentration of sample and TAC. For example, without wishing to be bound by theory, because amplification of the TAC is linear, such a linear IAC does not consume PCR reagents faster than that of an unknown sample when such a linear IAC is present at a higher concentration than that of an unknown sample. Moreover, when an IAC is present at a lower concentration than that of an unknown sample, such an amplicon may still be amplified as expected. IAC amplification may occur as expected because such IAC requires a lower amount of reagents for a linear amplification than it would if amplification of such an IAC was an exponential amplification. The present disclosure further encompasses a recognition that such (linear) internal amplification controls can be used to quantify and compare inhibition in reference and target samples.

Accordingly, in some embodiments, the present disclosure provides methods of performing nucleic acid amplification reactions, including steps of providing an internal amplification control (IAC), comprising a single oligonucleotide primer and a nucleic acid template; contacting an internal amplification control with a nucleic acid amplification reagent in a reaction vessel; and performing a nucleic acid amplification reaction, wherein a nucleic acid amplification template is linearly amplified, and wherein, when a target nucleic acid sample is present in a reaction vessel, a target nucleic acid sample is linearly or exponentially amplified.

In some embodiments, methods provided herein include steps such that when a single oligonucleotide primer binds with a nucleic acid template, a single oligonucleotide primer is extended by a polymerase. In some embodiments, provided methods include a step of activating a probe. In some embodiments, when a single oligonucleotide primer is extended by a polymerase, a probe activates. In some embodiments, when a probe activates, it produces a fluorescence signal. In some embodiments, provided methods include a step of quantifying an internal amplification control by its cycle threshold, slope, and/or end point fluorescence.

In some embodiments, a nucleic acid amplification reagent comprises target specific proteins. In some embodiments, a nucleic acid amplification reagent comprises a DNA polymerase at a concentration of at least about 8.0 U/reaction and a target specific primer concentration of at least about 1.5 μM, and an (IAC specific) single oligonucleotide primer concentration of at least about 5.0 μM.

In some embodiments, provided methods include a sequence of a nucleic acid template that is or comprises a single oligonucleotide primer or a sequence complementary to a single oligonucleotide primer. In some embodiments, a nucleic acid template is a plasmid that has more than one complementary sequence to the single oligonucleotide primer and the probe is a hydrolysis probe.

In some embodiments, provided methods include quantifying an internal amplification control in a target nucleic acid sample and a reference sample. In some embodiments, a target nucleic acid sample is an environmental sample.

In some embodiments, an internal amplification control (IAC) for a nucleic acid amplification reaction includes a single oligonucleotide primer and a nucleic acid template. In some embodiments, when present with a target nucleic acid sample and contacted with a nucleic acid amplification reagent in a reaction vessel, a nucleic acid template linearly amplifies and a target nucleic acid sample exponentially or linearly amplifies. In some embodiments, when a nucleic acid template is present at a higher concentration than a target nucleic acid sample, amplification of a nucleic acid template does not consume nucleic acid amplification reagents at a faster rate than amplification of a target nucleic acid sample.

In some embodiments, a nucleic acid amplification reagent includes a DNA polymerase at a concentration of at least about 8.0 U/reaction and a target specific primer concentration of at least about 1.5 μM, and an (TAC-specific) single oligonucleotide primer concentration of at least about 5.0 μM. In some embodiments, an internal amplification control includes a reference sample.

In some embodiments, a sequence of the nucleic acid template is or comprises a single oligonucleotide primer or a sequence complementary to a single oligonucleotide primer. In some embodiments, a nucleic acid template is a plasmid that has more than one complementary sequence to a single oligonucleotide primer. In some embodiments, when a single oligonucleotide primer binds to a nucleic acid template, a single oligonucleotide primer is extended by a polymerase.

In some embodiments, an internal amplification control includes a probe. In some embodiments, when a single oligonucleotide primer is extended by a polymerase, a probe activates. In some embodiments, a probe is a fluorescent probe. In some embodiments, a probe is a hydrolysis probe. In some embodiments, a probe produces a fluorescence signal. In some embodiments, when an internal amplification control is amplified its cycle threshold, slope or end point fluorescence is determined.

### BRIEF DESCRIPTION OF THE DRAWING

The foregoing and other objects, aspects, features, and advantages of the present disclosure will become more apparent and better understood by referring to the following description taken in conjunction with the accompanying figures in which:

FIG. 1 is a graph showing slopes of amplification plots in an example Taq enzymatic activity assay for six example samples.

FIG. 2 is a graph showing average cycle threshold (Ct) values from example Legionella (Lpn) qPCR cycling in six samples and a positive control.

FIG. 3 is a graph showing cycle threshold (Ct) values for example Legionella (Lpn) qPCR cycling in samples with different internal amplification control (IAC) DNA copy numbers.

FIG. 4 is a graph showing cycle threshold (Ct) values for example internal amplification control (IAC) DNA qPCR cycling in samples with different Legionella (Lpn) copy numbers.

FIG. 5 is a graph showing is a graph showing cycle threshold (Ct) values for example Legionella (Lpn) qPCR cycling in samples with or without internal amplification control (TAC) DNA.

### DEFINITIONS

In some embodiments, provided apparatus and/or methods are characterized in that they allow study of cell behavior in a variety of simulated biological environments and/or permit high-throughput analysis of cell attributes and/or responses, and/or those of agents that affect them. In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” As used in this application, the term “comprise” and variations of the term, such as “comprising” and “comprises,” are not intended to exclude other additives, components, integers or steps.

As used in this application, the terms “about” and “approximately” are used as equivalents. Any numerals used in this application with or without about/approximately are meant to cover any normal fluctuations appreciated by one of ordinary skill in the relevant art. In some embodiments, the term “approximately” or “about” refers to a range of values that fall within 25%, 20%, 19%, 18%, 17%, 16%, 15%, 14%, 13%, 12%, 11%, 10%, 9%, 8%., 7%, 6%, 5%, 4%, 3%, 2%, 1%, or less in either direction (greater than or less than) of the stated reference value unless otherwise stated or otherwise evident from the context (except where such number would exceed 100% of a possible value).

“Amplification” or “Amplify”: As used herein, the term “amplification” or “amplify” refers to methods known in the art for copying a target sequence from a template nucleic acid, thereby increasing the number of copies of the target sequence in a sample. Amplification may be exponential or linear. A template nucleic acid may be either DNA or RNA. The target sequences amplified in this manner form an “amplified region” or “amplicon.” While the exemplary methods described hereinafter relate to amplification using PCR, numerous other methods are known in the art for amplification of target nucleic acid sequences (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al. (1990). Eds. Academic Press, San Diego, Calif. . pp 13-20; Wharam et al. (2001). Nucleic Acids Res. 29(11): E54-E54; Hafner et al. (2001). Biotechniques. 30(4): 852-6, 858.860 passim. Further amplification methods suitable for use with the present methods include, for example, reverse transcription PCR (RT-PCR), ligase chain reaction (LCR), transcription-based amplification system (TAS), nucleic acid sequence based amplification (NASBA) reaction, self-sustained sequence replication (3SR), strand displacement amplification (SDA) reaction, boomerang DNA amplification (BDA). Q-beta replication, or isothermal nucleic acid sequence based amplification.

“Bacterial growth” or “Growth”: As used herein, the term “bacterial growth” or “growth” refers to a test result impacted by bacterial growth if the test value is at least 2-fold higher for a sample tested after a time delay (e.g., shipping delay of 1-3 days) as compared to a sample tested in parallel without a time delay.

“Bacterial degradation” or “Degradation”: As used herein, the term “bacterial degradation” or “degradation” refers to a test result impacted by bacterial degradation if the test value is at least 2-fold lower for a sample tested after a time delay (e.g., shipping delay of 1-3 days) as compared to a sample tested in parallel without a time delay.

“Biological sample”: As used herein, the term “biological sample” refers to a sample obtained from a biological source. In some embodiments, a biological sample is a body fluid sample (e.g., blood, cerebrospinal fluid, saliva, urine) or a cell sample. In some embodiments, a biological sample is a swab sample. In some embodiments, the biological sample is collected from a foodstuff or a mammal. In some embodiments, the mammal is a human.

“Colony forming units/milliliter”: As used herein, the term “colony forming units/milliliter” (CFU/mL) refers to a unit of measurement for estimating the number of bacterial cells grown on a bacterial plate.

“Corresponding to”: As used herein, the term “corresponding to” is often used to designate a structural element or moiety in an agent of interest that shares a position (e.g., in three-dimensional space or relative to another element or moiety) with one present in an appropriate reference agent. For example, in some embodiments, the term is used to refer to position/identity of a residue in a polymer, such as an amino acid residue in a polypeptide or a nucleotide residue in a nucleic acid. Those of ordinary skill will appreciate that, for purposes of simplicity, residues in such a polymer are often designated using a canonical numbering system based on a reference related polymer, so that a residue in a first polymer “corresponding to” a residue at position 190 in the reference polymer, for example, need not actually be the 190^{th} residue in the first polymer but rather corresponds to the residue found at the 190^{th} position in the reference polymer; those of ordinary skill in the art readily appreciate how to identify “corresponding” amino acids, including through use of one or more commercially-available algorithms specifically designed for polymer sequence comparisons.

“Direct qPCR”: As used herein, the term “direct qPCR” refers to methods comprising addition of a non-concentrated environmental sample directly into a qPCR system. Direct qPCR differs from Spartan qPCR and laboratory qPCR in that the environmental sample is not concentrated (e.g., by filtration) before analysis. In some embodiments, a LOD of direct qPCR is greater than 200 GU/mL. In some embodiments, a LOD of Spartan qPCR is less than 10 GU/mL. In some embodiments, a LOD of laboratory qPCR is less than 10 GU/mL.

“DNA”: As used herein, the term “DNA” refers to some or all of the DNA from a microorganism (e.g., bacteria, cyanobacteria, virus, protozoa, fungus, rotifer) or from the nucleus of a cell. DNA may be intact or fragmented (e.g., physically fragmented or digested with restriction endonucleases by methods known in the art). In some embodiments. DNA may include sequences from all or a portion of a single gene or from multiple genes. In some embodiments, DNA may be in the form of a plasmid. In some embodiments, DNA may be linear or circular. In some embodiments, DNA may include sequences from one or more chromosomes, or sequences from all chromosomes of a cell.

“Environmental Sample”: As used herein, the term “environmental sample” refers to a sample obtained from a non-biological source. In some embodiments, an environmental sample is an aqueous sample. e.g.. a water sample. In some embodiments, a water sample is obtained from an industrial, health-care or residential facility or setting. In some embodiments, a water sample is obtained from a natural setting (e.g., lake, stream, pond, reservoir, or other water source). In some embodiments, an environmental sample is a water sample obtained from an industrial cooling tower. In some embodiments, an environmental sample is a water sample obtained from an untreated fresh water source. In some embodiments, an environmental sample is a waste water sample. In some embodiments, an environmental sample is standing water (e.g., stagnant water), wash water or grey water. In some embodiments, an environmental sample is a water sample obtained from a lavatory, shower, bathtub, toilet, or sink.

“Fragment”: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole.

“Forward primer”: As used herein, the term “forward primer” refers to a primer that hybridizes to the anti-sense strand of dsDNA. A “reverse primer” hybridizes to the sense-strand of dsDNA.

“Genomic units/milliliter”: As used herein, the term “genomic units/milliliter” (GU/mL) refers to a unit of measurement for estimating the number of DNA copies (e.g., bacterial DNA copies) present in a sample. In some embodiments, GU/mL refers to “genomic equivalents/mL” or “GE/mL”.

“Hybridize” and “Hybridization”: As used herein, the terms “hybridize” and “hybridization” refer to a process where two complementary or partially-complementary nucleic acid strands anneal to each other as a result of Watson-Crick base pairing. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Those skilled in the art understand how to estimate and adjust the stringency of hybridization conditions such that sequences having at least a desired level of complementarities will form stable hybrids, while those having lower complementarities will not. For examples of hybridization conditions and parameters. see. e.g., Sambrook, et al., 1989, Molecular Cloning: A Laboratory Manual, Second Edition. Cold Spring Harbor Press, Plainview, N.Y.; Ausubel, F. M. et al. 1994, Current Protocols in Molecular Biology. John Wiley & Sons, Secaucus, N.J.

“Laboratory culture” or “Culture”: As used herein, the term “laboratory culture” or “culture” refers to the process of adding a sample to a nutrient-rich plate and allowing bacteria to grown in individual spots (colonies). In some embodiments, colonies are counted to determine the number of bacteria in a given sample (expressed as CFU/mL). Culture often involves pre-treatment of a sample to remove non-Legionella bacteria and antibiotic-treated culture plates to prevent growth of non-Legionella bacteria. In some embodiments. laboratory culture results are available by 10-14 days.

“Laboratory qPCR”: As used herein, the term “laboratory qPCR” refers to a method of concentrating bacteria, isolating their DNA, and quantifying the amount of DNA using qPCR. In some embodiments, laboratory qPCR is performed in accordance with ISO standard 12869:2012 “Water quality—Detection and quantification of Legionella ssp. and/or Legionella pneumophilia by concentration and genic amplification by quantitative polymerase chain reaction (qPCR).”

“Legionella pneumophila”: As used herein, the term “Legionella pneumophila” (L. pneumophilia) refers to a species of Legionella bacteria and is the primary causative agent of Legionnaires' disease. In some embodiments, there are 15 subtypes of L. pneumophilia that can be detected by methods described herein.

“Limit of Detection”: As used herein, the term “limit of detection” (LOD) refers to the lowest quantity of L. pneumophilia that is distinguishable from the absence of L. pneumophilia within the confidence limits of a method.

“Microorganism”: As used herein, the term “microorganism” refers to a microscopic organism that may be single-celled or multicellular. Examples of microorganisms include bacteria, cyanobacteria, viruses, protozoa, fungus and rotifers. In some embodiments, a bacterium is of the genus Alicyclobacillus, Aeromonas, Bacteroides, Bifidobacterium, Campvlobacter, Ciitrobacter, Clostridia, Enterobacter, Enteroccocus, Escherichia, Eubacterium, Klebsiella, Lactobacillus, Legionella, Listeria, Mycobactenum, Pseudomonas. Raoultella, Salmonella, Shigella, Streptococcus, Vibrio or a combination thereof. In some embodiments, the Legionella species is Legionella pneumophila, Legionella longbeachae, Legionella bozemannii, Legionella micdadei, Legionella feelei, Legionella dumoffii, Legionella wasdworthii or Legionella anisa. In some embodiments, the Escherichia species is Escherichia coli.

“Negative”: As used herein, the term “negative” refers to a test result, or group of test results, that comprise an undetectable level of L. pneumophilia, such as, a result below the LOD of the test.

“Nucleic acid”: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments. a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine. 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

“Positive”: As used herein, the term “positive” refers to a test result, or group of test results that comprise detectable levels of L. pneumophilia at or above the LOD of the test.

“Quantitative Polymerase Chain Reaction”: As used herein, the term “quantitative polymerase chain reaction” (qPCR) refers to a technology for amplifying sections of DNA. In some embodiments, quantitative PCR amplifies DNA and quantifies the amount of DNA. As used herein, the term “sense strand” refers to the strand of double-stranded DNA (dsDNA) that includes at least a portion of a coding sequence of a functional protein. “Anti-sense strand” refers to the strand of ds DNA that is the reverse complement of the sense strand.

“Reference”: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

“Sample”: As used herein, the term “sample” typically refers to a biological sample obtained or derived from a source of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids: sputum: saliva; urine: cerebrospinal fluid, peritoneal fluid; pleural fluid; feces; lymph; gynecological fluids; skin swabs; vaginal swabs; oral swabs: nasal swabs: washings or lavages such as a ductal lavages or bronchioalveolar lavages; aspirates; scrapings: bone marrow specimens; tissue biopsy specimens: surgical specimens; feces, other body fluids, secretions, and/or excretions: and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

“Spartan qPCR”: As used herein, the term “Spartan qPCR” is performed using methods described herein. In some embodiments, a method described herein is Spartan Legionella Detection System. In some embodiments, Spartan qPCR is completed within 2 hours, 1 hour, 45 minutes, 30 minutes or 15 minutes after collection of the sample from a source (e.g., an environmental source). In some embodiments, Spartan qPCR quantifies the amount of L. pneumophilia bacterial DNA (GU/mL) in a water sample (e.g., from an industrial cooling tower system).

“Swab sample”: As used herein, the term “swab sample” means a sample obtained with a collection tool. The collection tool may include a small piece of cotton or soft porous foam on the end of the tool, but is not required to. In general, a swab sample may be collected by contacting a sample source with a physical structure. Any physical structure that collects a swab sample when contacted with the sample source may be used for this purpose. In some embodiments, the physical structure may comprise an absorbent material (e.g., cotton). In some embodiments, the physical structure may be made of plastic and may collect the swab sample as a result of friction. In some embodiments, a swab sample is collected from a mammal (e.g., a human, dog, cat, cow, sheep, pig, etc.). In some embodiments, a mammal is a human. In some embodiments, a swab sample is collected from an open body cavity (e.g., mouth, nose, throat, ear, rectum, vagina, and wound). In some embodiments, a swab sample is a buccal sample. In some embodiments, a buccal sample may be collected by contacting (e.g., touching and/or swiping) the inside of a cheek. In some embodiments, a buccal sample may be collected by contacting with a tongue rather than a cheek. In some embodiments, a swab sample is collected from a body surface (e.g.. skin). In some embodiments. a swab sample is collected from the palm of a hand, inside the folds of the pinna of an ear, an armpit, or inside a nasal cavity. In some embodiments, a swab sample is collected from a foodstuff. In some embodiments, a foodstuff is raw. In some embodiments, a foodstuff is a fruit, a vegetable, a meat, a fish, or a shellfish. In some embodiments, meat is pork, beef, chicken or lamb. In some embodiments, a swab sample may be collected by touching and/or swiping the relevant foodstuff.

“Substantially”: As used herein, the term “substantially”, and grammatical equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Without Any Intervening Steps”: In some embodiments, the term “without any intervening steps” refers to directly contacting the nucleic acid amplification reagent with sample. For example, a concentrated sample comprising, for example, whole bacteria, cyanobacteria, virus, protozoa, fungus or rotifer. In some embodiments, a sample is a biological sample. In some embodiments, the term “without any intervening steps” comprises performing a method without steps such as lysing microorganisms present in a concentrated sample and/or purifying nucleic acids from microorganisms present in a concentrated sample. In some embodiments, the term “without any intervening steps” comprises performing a method without steps such as extracting or purifying nucleic acids present in a biological sample. Directly contacting may be achieved by, for example, placing the nucleic acid amplification reagent in a reaction vessel, then bringing the nucleic acid amplification reagent into contact with a sample (e.g., a concentrated environmental sample, a biological sample) by, for example, flicking the reaction vessel, inverting the reaction vessel, shaking the reaction vessel, vortexing the reaction vessel, etc.

### DETAILED DESCRIPTION OF CERTAIN EMBODIMENTS

Among other things, the present disclosure provides systems useful for amplifying nucleic acids. Various embodiments according to the present disclosure are described in detail herein. In particular, in some embodiments, the present disclosure provides systems and methods for performing a nucleic acid amplification reaction. In some embodiments, the present disclosure provides performing a nucleic acid amplification of a target sample in a presence of an internal amplification control. In some embodiments, the present disclosure provides performing a nucleic acid amplification of a target sample in a presence of an internal amplification control when an amount or concentration of an internal amplification control and/or a target sample is unknown. In some embodiments, the present disclosure provides performing a nucleic acid amplification of a target sample in a presence of an internal amplification control when an amount or concentration of an internal amplification control is greater than that of a target sample. In some embodiments, the present disclosure provides quantifying the amount of a target sample relative to a reference sample using an internal amplification control.

Implementations of the present disclosure are useful with a wide range of applications, including but not limited to: basic research, clinical medicine development. cloning, disease detection, disease diagnosis, forensic analysis, genetic mapping, genetic testing, identifying genetic mutation, industrial quality control, nucleic acid sequencing, tissue typing, environmental testing etc.

Nucleic acid amplification techniques vary in complexity and procedure but operate on the same general principle. Nucleic acid amplification techniques rapidly amplify specific regions, fragments, or portions of a nucleic acid sequence.

One skilled in the art will understand that numerous methods are known in the art for amplification of nucleic acids. Indeed, varied nucleic acid amplification techniques exist, see for example, Saiki,-Amplification of Genomic DNA” in PCR Protocols, Innis et al., Eds. Academic Press, San Diego, Calif., 13-20 (1990); see also Wharam et al. 29 Nucleic Acids Res. 11, E54-E54 (2001); see also Hafner et al., 30 Biotechniques 4, 852-6: 858, 860 passim (2001).

In some embodiments, amplification methods suitable for the present disclosure include, for example, boomerang DNA amplification (BDA), isothermal nucleic acid sequence based amplification, helicase dependent amplification (HDA), ligase chain reaction (LCR), loop mediated isothermal amplification, multiple displacement amplification, nucleic acid sequence based amplification (NASBA) reaction, polymerase chain reaction (PCR), Q-beta replication. reverse transcription PCR (RT-PCR), rolling circle amplification (RCA), self-sustained sequence replication (3SR), strand displacement amplification (SDA) reaction, transcription-based amplification system (TAS), or combinations thereof.

Nucleic acid amplification techniques typically include obtaining or collecting a sample of genetic material. The genetic material is contacted with nucleic acid amplification reaction mixture. The nucleic acid amplification reaction mixture involved in amplification methods, include, for example enzymes. primers, probes, buffers, etc. One skilled in the art will appreciate that these components and mixtures are readily available from commercial sources. for example, from Agilent Technologies, Bio-Rad, Biotools, Invitrogen, New England Biolabs, QIAGEN, R&D Systems, or Sigma-Aldrich, to name a few. One skilled in the art will also appreciate custom mixtures are and can be designed to address a specific or custom need.

As an example, PCR is one technique for making many copies of a specific target sequence within a template nucleic acid. PCR may be performed according to methods described in Whelan et al., 33 J. Clinical Microbiology, 3, 556-561 (1995). For example. a PCR reaction may consist of multiple amplification cycles and be initiated using a pair of primers that hybridize to the 5′ and 3′ ends of the target sequence. An amplification cycle may include an initial denaturation and typically up to 50 cycles of hybridization, strand elongation (or extension), and strand separation (denaturation). Hybridization and extension steps may be combined into a single step. In each cycle of a PCR reaction, a target sequence between primers is copied. Primers may hybridize to copied DNA amplicons as well as an original template DNA. A total number of copies increases exponentially with time/PCR cycle number.

Amplified target sequences or amplicons may be detected by any of a variety of well-known methods. For example, in some embodiments electrophoresis may be used (e.g., gel electrophoresis or capillary electrophoresis). Amplicons may also be subjected to differential methods of detection, for example, methods that involve the selective detection of variant sequences (e.g., detection of single nucleotide polymorphisms or SNPs using sequence specific probes). In some embodiments, amplicons are detected in real-time.

Sensitivity is a hallmark of nucleic acid amplification. Sensitivity refers to how effectively a sample is amplified. With respect to nucleic acid sequences, fragments, or portions thereof, nucleic acid techniques amplify anything and everything in a sample. This means that a nucleic acid technique can be used to find and amplify nucleic acids which may only be present in trace amounts in a sample.

The ability to amplify a tiny sample of nucleic acids can also mean that a limited sample can be compromised by inhibition. That is, these amplification techniques are very vulnerable to inhibition. Such inhibition is undesirable because it contributes to false, incorrect, or inaccurate test results.

A common problem of real-time PCR assays is failure of DNA amplification due to inhibitory substances in the samples. To detect inhibition, competitive and non-competitive internal amplification controls (IACs) are often used. For example, Randall et al. (2010) developed competitive and non-competitive IACs for a real-time PCR assay for Campylobacter coli and Campylobacter jejuni. (Randall L et al. (2010). Development and evaluation of internal amplification controls for use in a real-time duplex PCR assay for detection of Campylobacter coli and Campylobacter jejuni. Journal of Medical Microbiology. 59: 172-178.). Both of these TACs are exponentially amplified during each cycle of real-time PCR.

A limitation of both of these types of IACs is that they can interfere with detection or quantification of an unknown sample by quantitative PCR (qPCR). For example, if the IAC is present at a higher concentration than the unknown sample, the IAC may consume PCR reagents faster and lead to a delayed cycle threshold (Ct) value for the unknown sample. This could lead to an underestimate of the quantity of the unknown sample. For example, Randall et al. (2010) noted that “The FV-IAC caused inhibition of the PCR assay at dilutions of 10exp-5 and above.”

Conversely, if an IAC is present at a lower concentration than the unknown sample, the unknown sample may consume PCR reagents faster and lead to a delayed or even non-existent cycle threshold (Ct) value for the IAC. This could lead the user to invalidate the reaction due to failure of the IAC, even though the reaction was actually performing correctly. For example, Randall et al. (2010) noted that “The 16S rDNA IAC signals were generally lost in the presence of signals (unless Ct values were very high indicating weak signal) for either the C. coli or C. jejuni components of the PCR assay.”

Similar to Randall et al. (2010). Oikonomou et al. (2008) stated that there are issues with traditional competitive and non-competitive IACs. With the competitive strategy, “the amount of IAC is critical to the detection limit of the target as there is always some competition between target DNA and TAC” and “High concentration of IAC can abolish the target signal because of competition and cause false negative results, especially if the target is present in extremely low concentration.” With the non-competitive strategy. “the IAC are amplified using a different set of primers for each one. The development of two different PCRs, optimized to work under the same PCR conditions, may become sub-efficient for one or both reactions”. To solve these issues. Oikonomou developed a novel IAC strategy involving “a large size difference between the IAC (3196 bp) and the target (274 bp)” and initial cycling amplification with an extension time of 30 sec followed by cycling amplification with an extension time of 3 min. This process selectively enriches the target at the beginning of the reaction and allows it to consistently outcompete the IAC for PCR reagents even if the target is present in small quantities relative to the IAC. One disadvantage of Oikonomou's approach is that the IAC may not be amplified if the target is present in large quantities. Oikonomou states: “When the target DNA is amplified but the IAC is not, the positive result is valid because the TAC amplification is unnecessary”. While this may be true if the reaction is meant to provide a qualitative result, this could be a disadvantage if one wishes to use the IAC as an indicator of inhibitory effects on a quantitative reaction. For example, if the IAC has a known end-point fluorescence in a non-inhibited reaction, but this end-point fluorescence is lowered in a reaction with an unknown sample, then this indicates inhibitory effects on the quantification of the unknown sample. Another disadvantage is that the increased extension time increases the overall time of the reaction.

In some embodiments, the present disclosure provides an IAC that is amplified linearly rather than exponentially during qPCR. In some embodiments, because amplification is linear, such an TAC does not consume PCR reagents faster than that of an unknown sample when such an IAC is present at a higher concentration than that of an unknown sample. Moreover, when an IAC is present at a lower concentration than that of an unknown sample, such an amplicon is still amplified as expected. In some embodiments, IAC amplification when an IAC is present at a lower concentration is as expected because such IAC requires a lower amount of reagents for a linear amplification than it would if amplification of such an IAC was an exponential amplification.

In some embodiments, a linearly-amplified IAC is amplified in a presence of a Molecular Beacon probe and one primer that is complementary to a portion of a linearly-amplified IAC. In some embodiments, since there is only one primer, no new templates are formed and amplification is linear with each cycle. In some embodiments, during each cycle of qPCR, a single primer anneals to a probe and is extended by a DNA polymerase. In some embodiments, a Molecular Beacon opens and allows fluorescence to be measured.

In some embodiments, a linearly-amplified IAC is amplified in a presence of a circular plasmid having repeating sequences that ar complementary with a primer sequence and a Taqman probe sequence. In some embodiments, during each cycle of qPCR, a primer and Taqman probe bind to one of those repeating sequences. In some embodiments. a primer is extended by a DNA polymerase and this leads to probe displacement and cleavage. In some embodiments, resulting fluorescence may be measured.

In some embodiments, when a qualitative nucleic acid amplification reaction is performed with an IAC as disclosed herein, an IAC will be amplified successfully (e.g.. the IAC is detectable, e.g., at any Ct) if it is present in a small quantity relative to an unknown sample (e.g.. ratios of IAC copy number:sample genomic units of about 1:10; about 1:20; about 1:30; about 1:40; about 1:50: about 1:60; about 1:70; about 1:80: about 1:90; about 1:100; about 1:200; about 1:300: about 1:400: about 1:500; about 1:600; about 1:700; about 1:800; about 1:900; about 1:1000; or between about 1:1 and 1:1000; between about 1:2 and 1:500; between about 1:5 and 1:250; between about 1:10 and 1:100; between about 1:1 and 1:10; between about 1:1 and 1:20; between about 1:1 and 1:50; or between about 1:1 and 1:100). In some embodiments, an IAC will be amplified successfully (e.g., the IAC is detectable, e.g., at any Ct) if it is present in a quantity relative to an unknown sample (e.g., ratios of IAC copy number:sample genomic units) of about 1000:1; about 500:1: about 100:1; about 50:1; about 20:1; or about 1:1; or between about 1000:1 and 100:1: between about 100:1 and 10:1; or between about 10:1 and 1.1. In some embodiments, IAC will be amplified successfully if the ratio of IAC copy number:sample genomic units is at least about 0.001, about 0.005, about 0.01, about 0.05, about 0.1, about 0.5, about 1.0, about 1.5; about 2.0, about 2.5, about 5.0, about 10.0, about 15.0, about 20.0, about 50.0. about 100.0, about 500.0, or about 1000.0. In some embodiments, an IAC will not outcompete an unknown target sample even if such an IAC is present in a large quantity relative to an unknown.

In some embodiments, when a qualitative nucleic acid amplification reaction is performed with an IAC as disclosed herein, a sample will be amplified successfully (e.g., the sample is detectable, e.g., at any Ct) if it is present in a small quantity relative to an IAC (e.g., ratios of sample genomic units:IAC copy number of about 1:10: about 1:20; about 1:30; about 1:40; about 1:50; about 1:60; about 1:70; about 1:80; about 1:90; about 1:100; about 1:200; about 1:300; about 1:400; about 1:500; about 1:600: about 1:700: about 1:800; about 1:900; about 1:1000; or between about 1:1 and 1:1000; between about 1:2 and 1:500; between about 1:5 and 1:250; between about 1:10 and 1:100; between about 1:1 and 1:10; between about 1:1 and 1:20: between about 1:1 and 1:50, between about 1:1 and 1:100, or between about 1:1 and 1:1000). In some embodiments, a sample will be amplified successfully (e.g., the sample is detectable, e.g., at any Ct) if it is present in a quantity relative to an IAC (e.g., ratios of sample genomic units:IAC copy number) of about 1000:1; about 500:1; about 100:1; about 50:1; about 20:1; or about 1:1; or between about 1000:1 and 100:1; between about 100:1 and 10:1; between about 10:1 and 1.1. In some embodiments, a sample will be amplified successfully if the sample genomic units:IAC copy number is at least about 0.001, about 0.005, about 0.01, about 0.05, about 0.1, about 0.5, about 1.0, about 1.5; about 2.0, about 2.5, about 5.0, about 10.0, about 15.0, about 20.0, about 50.0, about 100.0, about 500.0, or about 1000.0.

In some embodiments, when performing a quantitative reaction, an IAC as disclosed herein can be used to assess inhibitory effects of an unknown sample. For example, one can determine an IAC's cycle threshold (Ct) that is generated with a non-inhibited sample and then compare it with a Ct that is generated with an unknown sample. In some embodiments, if there is a difference in a Ct, then such a difference indicates inhibitory effects. In some embodiments, a magnitude of difference in an IAC's Ct could be used to correct for a quantification value for an unknown target.

In some embodiments, when performing a quantitative reaction, an IAC as disclosed herein can be used to assess inhibitory effects of an unknown sample. For example, one can determine an IAC's slope that is generated with a non-inhibited sample and then compare the slope to that generated with an unknown sample. In some embodiments, if there is a difference in a slope, then such a difference indicates inhibitory effects. In some embodiments, a magnitude of difference in an IAC's Ct slope could be used to correct for a quantification value for an unknown target.

In some embodiments, when performing a quantitative reaction, an TAC as disclosed herein can be used to assess inhibitory effects of an unknown sample. For example, one can determine an IAC's end point fluorescence that is generated with a non-inhibited sample and then compare it with an end point fluorescence that is generated with an unknown sample. In some embodiments, if there is a difference in an end point fluorescence, then such a difference indicates inhibitory effects. In some embodiments, a magnitude of difference in an IAC's end point fluorescence could be used to correct for a quantification value for an unknown target.

In some embodiments, when performing a quantitative reaction, an IAC as disclosed herein can be used to assess inhibitory effects of an unknown sample. For example, one can determine an IAC's fluorescence that is generated with a non-inhibited sample at the beginning of a reaction and compare it with fluorescence that is generated with an unknown sample at the beginning of a reaction. In some embodiments, if there is a difference in an end point fluorescence, then such a difference indicates inhibitory effects. In some embodiments, a magnitude of difference in an IAC's end point fluorescence could be used to correct for a quantification value for an unknown target.

### Concentration of Microorganisms

As detailed herein, a sample, which may be an environmental sample, is collected and microorganisms present in the sample are concentrated. Concentration of the microorganisms present in the sample comprises removal and/or reduction of an aqueous component of the sample to produce a “concentrated sample.” In some embodiments, a concentrated sample comprises an increased concentration, level, percentage and/or amount of microorganism as compared to the environmental sample.

Concentration of microorganisms in a sample may be performed without lysis of the microorganism. Concentration of a microorganism in a sample may be performed without release, extraction and/or purification of the nucleic acid from the microorganism.

In some embodiments, a sample may be concentrated by filtration, for example using a filter membrane. In some embodiments, a filter membrane is hydrophilic. In some embodiments, a filter membrane is a hydrophilic polyethersulfone (PES) filter. In some embodiments, filtration comprises a step of washing a retentate and/or eluting a concentrated sample from the filter. In some embodiments, washing is performed using a buffer comprising water, 1X GoTaq colorless buffer (Promega, Cat. No. M7921), 2.5 mM magnesium chloride, 0.1% w/v sodium azide, and 0.05% w/v sodium hexametaphosphate. In some embodiments, a wash buffer is phosphate buffered saline. A volume of wash buffer used to wash a retentate may vary depending upon the amount environmental sample that is filtered. In some embodiments about 1 mL, about 2 mL, about 3 mL, about 4 mL, about 5 mL, about 6 mL, about 7 mL, about 8 mL, about 9 mL, about 10 mL or more of wash buffer is used. In some embodiments, a volume of wash buffer is 2 mL. A washing step may be performed one or more times.

In some embodiments, a concentrated sample may be eluted from a filter membrane. Elution of a concentrated sample may be performed using a buffer that is the same, or similar to a wash buffer. For example, an elution buffer may comprise water, 1X GoTaq colorless buffer (Promega, Cat. No. M7921), 2.5 mM magnesium chloride. 0.1% w/v sodium azide, and 0.05% w/v sodium hexametaphosphate. In some embodiments, an elution buffer is phosphate buffered saline. A volume of elution buffer used to elute a retentate from a filter may vary depending on the degree of concentration to be achieved. In some embodiments, a volume of elution buffer is about 100 μL, about 200 μL, about 300 μL, about 400 μL, about 500 μL about 600 μL, about 700 μL, about 800 μL, about 900 μL about 1 mL, about 2 mL, about 5 mL or more. An elution buffer may be contacted with a filter membrane one or more times. For example, an elution buffer may be pulsed back and forth across a membrane multiple times in order to elute a retentate and produce a concentrated sample. In some embodiments, an elution buffer is pulsed back and forth across a membrane about 5, about 10, about 15, about 20, about 25, about 50 times or more to elute a retentate and produce a concentrated sample. In some embodiments, an elution buffer is pulsed back and forth across a membrane about 20 times.

In some embodiments, an environmental sample is concentrated by evaporation and/or centrifugation.

In some embodiments, a sample is concentrated about 0.5-fold, 2-fold, 3-fold, 4-fold, 5-fold, 6-fold, 7-fold, 8-fold. 9-fold, 10-fold, 15-fold, 20-fold, 25-fold, 30-fold, 35-fold. 40-fold, 50-fold, 60-fold, 70-fold, 80-fold, 90-fold, 100-fold, 125-fold, 150-fold, 175-fold, 200-fold, 300-fold, 400-fold, 500-fold, 600-fold or ranges within as compared to an environmental sample. In some embodiments, a sample is concentrated about 500-fold as compared to an environmental sample. In some embodiments, a sample is concentrated about 375-fold as compared to an environmental sample. In some embodiments, a sample is concentrated about 250-fold as compared to an environmental sample. In some embodiments, a sample is concentrated about 125-fold as compared to an environmental sample. In some embodiments, a sample is concentrated about 63-fold as compared to an environmental sample. In some embodiments, a sample is concentrated about 31-fold as compared to an environmental sample. In some embodiments, a sample is concentrated about 16-fold as compared to an environmental sample. In some embodiments, a sample is concentrated about 8-fold as compared to an environmental sample. In some embodiments, a sample is concentrated about 0.5-fold as compared to an environmental sample.

In some embodiments, an environmental sample may be concentrated within a range. For example. from about 0.5-fold to about 500-fold as compared to an environmental sample. In some embodiments, a sample may be concentrated by about 8-fold to about 375-fold as compared to an environmental sample. In some embodiments, a sample may be concentrated by about 16-fold to about 250-fold as compared to an environmental sample. In some embodiments, a sample may be concentrated by about 31-fold to about 125-fold as compared to an environmental sample. In some embodiments, a sample may be concentrated by about 16-fold to about 31-fold as compared to an environmental sample. In some embodiments, a sample may be concentrated by about 8-fold to about 63-fold as compared to an environmental sample. In some embodiments, a sample may be concentrated by about 2-fold to about 125-fold as compared to an environmental sample.

In some embodiments, microorganisms present in an environmental sample may be lysed prior to concentration of the sample. In some embodiments, lysis may be performed using a surfactant (e.g., an anionic surfactant, an ionic surfactant). In some embodiments, a surfactant is an anionic surfactant (e.g., SDS). In some embodiments, a surfactant concentration in an amplification reaction is less than or equal to about 0.005% (w/v). In some embodiments, lysis may be performed using thermal treatment (e.g., high heat).

A concentrated sample may be directly contacted with a nucleic acid amplification reagent in a reaction vessel without any intervening steps. In some embodiments, the nucleic acid amplification reagent is directly contacted with a concentrated sample comprising, for example, whole bacteria, cyanobacteria, virus, protozoa, fungus or rotifer. In some embodiments, a method without any intervening steps is performed without steps such as lysing microorganisms present in a concentrated sample and/or purifying nucleic acids from microorganisms present in a concentrated sample. Directly contacting may be achieved by, for example, placing a nucleic acid amplification reagent in a reaction vessel, then bringing the nucleic acid amplification reagent into contact with the concentrated sample (e.g., by flicking the reaction vessel, inverting the reaction vessel, shaking the reaction vessel, vortexing the reaction vessel, etc.).

### Amplification of Nucleic Acids

In various embodiments, template nucleic acids from the sample may be amplified using polymerase chain reaction (PCR) or reverse transcription PCR (RT-PCR); however, as noted previously, the skilled artisan will understand that numerous methods are known in the art for amplification of nucleic acids, and that these methods may be used either in place of, or together with, PCR or RT-PCR. For example, without limitation, other amplification methods employ ligase chain reaction (LCR), transcription-based amplification system (TAS). nucleic acid sequence based amplification (NASBA) reaction, self-sustained sequence replication (3SR), strand displacement amplification (SDA) reaction, boomerang DNA amplification (BDA), Q-beta replication, isothermal nucleic acid sequence based amplification, etc. In general, nucleic acid amplification methods, such as PCR, RT-PCR, isothermal methods, rolling circle methods, etc., are well known to the skilled artisan. See, e.g., Saiki, “Amplification of Genomic DNA” in PCR Protocols, Innis et al. (1990). Eds. Academic Press, San Diego, Calif.. pp 13-20; Wharam et al. (2001). Nucleic Acids Res. 29(11): E54-E54; Hafner et al. (2001). Biotechniques. 30(4): 852-6, 858, 860 passim.

The nucleic acid amplification reagents that are involved in each of these amplification methods (e.g., enzymes, primers, probes, buffers. surfactants etc.) may vary but are also well known in the art and readily available from commercial sources (e.g., see catalogues from Invitrogen, Biotools, New England Biolabs, Bio-Rad, QIAGEN, Sigma-Aldrich, Agilent Technologies, R&D Systems. etc.). It will also be appreciated that the specific primers and/or probes that are used in any given method will depend on the template nucleic acid and the target sequence that is being amplified and that those skilled in the art may readily design and make suitable primers and/or probes for different template nucleic acids and target sequences. Primers and probes may also be prepared by commercial suppliers (e.g., Integrated DNA Technologies).

In some embodiments, a nucleic acid amplification reaction of the methods described herein may contain DNA polymerase at a concentration substantially higher than typically used in standard amplification reactions (e.g., higher than 1.0 U/20 μL reaction). In some embodiments, the sample matrix (e.g., HVAC concentrate) may be an inhibitory environment for, e.g.. DNA polymerase activity; accordingly, in some embodiments, a higher concentration of reagent (e.g., DNA polymerase, e.g., Taq polymerase) may help overcome any such reaction inhibition. Moreover, use of relatively high reagent concentration may help detect target DNA, particularly when present at very low concentrations. In the embodiments disclosed herein, the reaction volume is typically 20 μL. Those skilled in the art, reading the present specification, will appreciate that when the reaction volume is larger or smaller than 20 μL, the amount of DNA polymerase used in the reaction is adjusted accordingly. In some embodiments, a DNA polymerase concentration is at least 1.0 U/reaction, e.g., at least 1.2 U/reaction, at least 1.4 U/reaction, at least 1.6 U/reaction, at least 1.8 U/reaction, at least 2.0 U/reaction, at least 2.2 U/reaction, at least 2.4 U/reaction, at least 2.6 U/reaction, at least 2.8 U/reaction, at least 3.0 U/reaction, at least 3.2 U/reaction, at least 3.4 U/reaction, at least 3.6 U/reaction, at least 3.8 U/reaction, at least 4.0 U/reaction, at least 5.0 U/reaction, at least 6.0 U/reaction, at least 7.0 U/reaction, at least 8.0 U/reaction, at least 9.0 U/reaction, at least 10 U/reaction, at least 11 U/reaction, at least 12 U/reaction, at least 13 U/reaction, at least 14 U/reaction, at least 15 U/reaction, at least 20 U/reaction, at least 25 U/reaction, at least 30 U/reaction, at least 25 U/reaction, at least 30 U/reaction, at least 35 U/reaction, at least 40 U/reaction, at least 45 U/reaction, at least 50 U/reaction or higher. In some embodiments, a DNA polymerase concentration is 3.4 U/reaction. In some embodiments, a DNA polymerase concentration is 6 U/reaction. In some embodiments, a DNA polymerase concentration is 12 U/reaction. In some embodiments, a DNA polymerase concentration is 21 U/reaction. In some embodiments, a DNA polymerase concentration is 42 U/reaction. In some embodiments, a DNA polymerase concentration ranges from at least 3.4 U/reaction to about 45 U/reaction. In some embodiments, a DNA polymerase concentration ranges from at least 12 U/reaction to about 21 U/reaction. In some embodiments, a DNA polymerase concentration ranges from at least 6 U/reaction to about 42 U/reaction.

In some embodiments, a nucleic acid amplification reaction may contain target primer concentrations substantially higher than typically used in standard amplification reactions (e.g., concentrations higher than 0.1-0.2 μM). In some embodiments, higher target primer concentrations may be used, e.g., to accelerate PCR cycling, to induce and/or improve higher temperature PCR, to increase reaction efficiencies, to help overcome matrix inhibition, and/or to enable or improve detection at low target concentrations. In some embodiments, a target primer concentration in an amplification reaction is at least 0.1 μM, e.g., at least 0.2 μM, at least 0.4 μM, at least 0.6 μM, at least 0.8 μM, at least 1.0 μM, at least 1.2 μM, at least 1.4 μM, at least 1.6 μM, at least 1.8 μM, at least 2.0 μM, at least 2.5 μM, at least 3.0 μM, at least 3.5 μM, at least 4.0 μM, at least 4.5 μM, at least 5.0 μM, at least 5.5 μM, at least 6.0 μM, at least 6.5 μM, at least 7.0 μM, at least 7.5 μM, at least 8.0 μM, at least 8.5 μM, at least 9.0 μM, at least 9.5 μM, at least 10 μM. at least 11 μM, at least 12 μM, at least 13 μM. at least 14 μM. at least 15 μM or higher. In some embodiments, a target primer concentration in an amplification reaction is at least 1.3 μM. In some embodiments, a target primer concentration in an amplification reaction is at least 2.0 μM. In some embodiments, a target primer concentration in an amplification reaction is at least 4.0 μM. In some embodiments, a target primer concentration in an amplification reaction is at least 7.0 μM. In some embodiments, a target primer concentration in an amplification reaction is at least 14 μM. In some embodiments, a target primer concentration in an amplification reaction ranges from at least 1.3 μM to about 15 μM. In some embodiments. a target primer concentration in an amplification reaction ranges from at least 4 μM to about 7 μM. In some embodiments, a target primer concentration in an amplification reaction ranges from at least 2 μM to about 14 μM. It is to be understood that these values refer to the concentration of each primer (e.g., the concentration of the forward target primer or the target reverse primer) used in the reaction. In some embodiments, a forward target primer concentration in an amplification reaction is 1.3 μM. In some embodiments, a reverse target primer concentration in an amplification reaction is 1.3 μM.

In some embodiments, a nucleic acid amplification reaction may contain (IAC specific) single oligonucleotide primer concentrations substantially higher than typically used in amplification reactions (e.g., 0.1-0.2 μM). In some embodiments, higher single oligonucleotide primer concentrations may be used, e.g., to accelerate PCR cycling, to induce and/or improve higher temperature PCR, to increase reaction efficiencies, to help overcome matrix inhibition. and/or to enable or improve detection/signal strength of an IAC probe. In some embodiments, a single oligonucleotide primer concentration in an amplification reaction is at least 0.1 μM, e.g., at least 0.2 μM, at least 0.4 μM, at least 0.6 μM, at least 0.8 μM, at least 1.0 μM, at least 1.2 μM, at least 1.4 μM, at least 1.6 μM, at least 1.8 μM, at least 2.0 μM, at least 2.5 μM, at least 3.0 μM, at least 3.5 μM, at least 4.0 μM, at least 4.5 μM, at least 5.0 μM, at least 5.5 μM, at least 6.0 μM, at least 6.5 μM, at least 7.0 μM, at least 7.5 μM, at least 8.0 μM, at least 8.5 μM, at least 9.0 μM, at least 9.5 μM, at least 10 μM, at least 11 μM, at least 12 μM, at least 13 μM, at least 14 μM, at least 15 μM or higher. In some embodiments, a single oligonucleotide primer concentration in an amplification reaction is at least 1.3 μM. In some embodiments, a single oligonucleotide primer concentration in an amplification reaction is at least 2.0 μM. In some embodiments, a single oligonucleotide primer concentration in an amplification reaction is at least 4.0 μM. In some embodiments, a single oligonucleotide primer concentration in an amplification reaction is at least 7.0 μM. In some embodiments, a single oligonucleotide primer concentration in an amplification reaction is at least 14 μM. In some embodiments, a single oligonucleotide primer concentration in an amplification reaction ranges from at least 1.3 μM to about 15 μM. In some embodiments, a single oligonucleotide primer concentration in an amplification reaction ranges from at least 4 μM to about 7 μM. In some embodiments, a single oligonucleotide primer concentration in an amplification reaction ranges from at least 2 μM to about 14 μM. It is to be understood that these values refer to the concentration of each single oligonucleotide primer (e.g., the concentration of the forward single oligonucleotide primer or the reverse single oligonucleotide primer) used in the reaction. In some embodiments, a forward single oligonucleotide primer concentration in an amplification reaction is 1.3 μM. In some embodiments, a reverse single oligonucleotide primer concentration in an amplification reaction is 1.3 μM.

In some embodiments, a nucleic acid amplification reaction may contain probe concentrations substantially higher than typically used in standard amplification reactions (e.g., higher than 0.1-0.2 μM). In some embodiments, higher probe concentrations may be used e.g., to accelerate PCR cycling, to induce and/or improve higher temperature PCR, to increase reaction efficiencies, to help overcome matrix inhibition, and/or to enable or improve detection at low target concentrations/signal strength. In some embodiments, a probe concentration in a nucleic acid amplification reaction is at least 0.2 μM, e.g., at least 0.3 μM, at least 0.4 μM, at least 0.5 μM, at least 0.6 μM, at least 0.7 μM, at least 0.8 μM, at least 0.9 μM, at least 1.0 μM, at least 1.2 μM, at least 1.4 μM, at least 1.5 μM, at least 1.6 μM, at least 1.8 μM, at least 2.0 μM, at least 3.0 μM, at least 4.0 μM, at least 5.0 μM, at least 6.0 μM, at least 7.0 μM, at least 8.0 μM, at least 9.0 μM, at least 10 μM, at least 11 μM, at least 12 μM, at least 13 μM, at least 14 μM, at least 15 μM or higher. In some embodiments, a probe concentration in an amplification reaction is at least 1.0 μM. In some embodiments, a probe concentration in an amplification reaction is at least 1.95 μM. In some embodiments, a probe concentration in an amplification reaction is at least 3.9 μM. In some embodiments, a probe concentration in an amplification reaction is at least 6.8 μM. In some embodiments, a probe concentration in an amplification reaction is at least 13.7 μM. In some embodiments, a probe concentration ranges from at least 1.0 μM to about 14 μM. In some embodiments, a probe concentration ranges from at least 3.5 μM to about 7.0 μM. In some embodiments, a probe concentration ranges from at least 1.9 μM to about 14 ρM. It is to be understood that these values refer to the concentration of each probe (e.g., a concentration of a mutant probe or a wild-type probe) in an amplification reaction.

In some embodiments, a nucleic acid amplification reaction may contain deoxynucleotides (dNTP) concentrations substantially higher than typically used in amplification reactions (e.g., 0.1-0.2 mM). In some embodiments, a dNTP concentration in a nucleic acid amplification reaction is at least 0.2 mM, e.g., at least 0.3 mM, at least 0.4 mM, at least 0.5 mM, at least 0.6 mM, at least 0.7 mM, at least 0.8 mM, at least 0.9 mM, at least 1.0 mM, at least 1.2 mM, at least 1.4 mM, at least 1.6 mM, at least 1.8 mM, at least 2.0 mM, at least 2.2 mM, at least 2.4 mM, at least 2.6 mM, at least 2.8 mM, at least 3.0 mM or higher. In some embodiments, a dNTP concentration in an amplification reaction is at least 0.3 mM. In some embodiments, a dNTP concentration in an amplification reaction is at least 0.6 mM. In some embodiments, a dNTP concentration in an amplification reaction is at least 1.05 mM. In some embodiments, a dNTP concentration in an amplification reaction is at least 2.1 mM.

In some embodiments, a single oligonucleotide primer concentration in a nucleic acid amplification reaction is at least 0.5 μM and a probe concentration is at least 0.7 μM. In some embodiments, an amplification reaction comprises a forward single oligonucleotide primer at a concentration of 1.3 μM, a reverse single oligonucleotide primer at a concentration of 1.3 μM and a probe at a concentration of 1 μM.

In some embodiments, a nucleic acid amplification reaction contains DNA polymerase, target primer, a single oligonucleotide primer, and probe concentrations substantially higher than typically used in amplification reactions. In some embodiments, an amplification reaction comprises a DNA polymerase concentration of 3.4 U/reaction, a primer concentration of 1.3 μM and a probe concentration of 1.0 μM.

In some embodiments, an amplification reaction comprises a DNA polymerase concentration ranging from at least 3.4 U/reaction to about 45 U/reaction, a primer concentration ranging from at least 1.3 μM to about 15 μM and a probe concentration ranging from at least 1.0 μM to about 14 μM. In some embodiments, an amplification reaction comprises a DNA polymerase concentration ranging from at least 12 U/reaction to about 21 U/reaction, a primer concentration ranging from at least 4 μM to about 7 μM and a probe concentration ranging from at least 3.5 μM to about 7 μM. In some embodiments, an amplification reaction comprises a DNA polymerase concentration ranging from at least 6 U/reaction to about 42 U/reaction, a primer concentration ranging from at least 2 μM to about 14 μM and a prob^{e} concentration ranging from at least 1.9 μM to about 14 μM.

In some embodiments, a nucleic acid amplification reaction comprises a surfactant (e.g., an anionic surfactant, an ionic surfactant). In some embodiments, a surfactant is an anionic surfactant (e.g., SDS). In some embodiments, a surfactant concentration in an amplification reaction is less than or equal to about 0.005% (w/v). In some embodiments, microorganisms present in a concentrated sample may be lysed following contact with a nucleic acid amplification reagent and heating.

PCR is a technique for making many copies of a specific target sequence within a template DNA. The reaction consists of multiple amplification cycles and is initiated using a pair of primer oligonucleotides that hybridize to the 5′ and 3′ ends of the target sequence. The amplification cycle includes an initial denaturation and typically up to 50 cycles of hybridization, strand elongation (or extension), and strand separation (denaturation). The hybridization and extension steps may be combined into a single step. In each cycle of the reaction, the target sequence between the primers is copied. Primers may hybridize to the copied DNA amplicons as well as the original template DNA, so the total number of copies increases exponentially with time/PCR cycle number. In some embodiments, PCR may be performed according to methods described in Whelan et al. (J. Clin. Microbiol (1995) 33(3):556-561). Briefly, the nucleic acid amplification reagents (PCR reaction mixture) include two specific primers per target sequence, dNTPs, a DNA polymerase (e.g., Taq polymerase), and a buffer (e.g., 1X PCR Buffer). The amplification reaction itself is performed using a thermal cycler. Cycling parameters may be varied, depending on, for example, the melting temperatures of the primers or the length of the target sequence(s) to be extended. As mentioned previously, the skilled artisan is capable of designing and preparing primers that are appropriate for amplifying a target sequence. The length of the amplification primers for use in the present methods depends on several factors including the level of nucleotide sequence identity between the primers and complementary regions of the template nucleic acid and also the temperature at which the primers are hybridized to the template nucleic acid. The considerations necessary to determine a preferred length for an amplification primer of a particular sequence identity are well-known to a person of ordinary skill in the art and include considerations described herein. For example, the length and sequence of a primer may relate to its desired hybridization specificity or selectivity.

In some embodiments, an environmental sample (optionally a concentrated sample) is contacted with a nucleic acid amplification reagent right after collection of the sample, for example, within about 1-30 minutes of collection. In some embodiments, an environmental sample (optionally a concentrated sample) is contacted with a nucleic acid amplification reagent within about 1 to 60 minutes, within about 1 hour to 8 hours, within about 8 hours to 24 hours, within about 1 day to 3 days, or within about 5 days of collection.

In some embodiments, a nucleic acid amplification reaction is performed within 120 minutes of contacting an environmental sample (optionally a concentrated sample) with a nucleic acid amplification reagent. In some embodiments, the nucleic acid amplification reaction is performed even sooner, e.g., within 60, 30, 15, 10, 5 or even 1 minute(s) of contacting a concentrated sample with the nucleic acid amplification reagent.

In some embodiments, a nucleic acid amplification reaction is completed within 120 minutes of contacting a concentrated sample with a nucleic acid amplification reagent. In some embodiments, the nucleic acid amplification reaction is completed even sooner, e.g., within 60, 30, 15, 10, 5 or even 1 minute(s) of contacting a concentrated sample with the nucleic acid amplification reagent.

In some embodiments, a nucleic acid amplification reaction comprises an initial heat denaturation step of 15 minutes or less. In some embodiments, an initial heat denaturation step is shorter, e.g., 5 minutes or less, 3 minutes or less, 1 minute or less or 30 seconds or less. In some embodiments, an initial heat denaturation is 4.5 minutes. In some embodiments, an initial heat denaturation step is performed at a temperature in the range of about 85° C. to about 105° C. e.g., about 93° C. to about 97° C. about 93° C. to about 95° C., or about 95° C. to about 97° C., etc. In some embodiments, an initial heat denaturation step is performed at about 95° C. In some embodiments, an initial heat denaturation step is performed at about 99° C. In some embodiments an initial heat denaturation step is performed at about 99° C. to about 101° C. In some embodiments, an initial heat denaturation step is performed at about 101° C. to about 103° C.

In some embodiments, an initial heat denaturation step is performed at more than one temperature, for example, at a first temperature followed by a second temperature. In some embodiments, a first temperature is in the range of about 85° C. to about 105° C., e.g., about 93° C. to about 97° C., about 93° C. to about 95° C., or about 95° C. to about 97° C., etc. In some embodiments a second temperature is in the range of about 85° C. to about 105° C., e.g., about 93° C. to about 97° C., about 93° C. to about 95° C., or about 95° C. to about 97° C., etc. In some embodiments, the initial heat denaturation step comprises a first temperature of about 98° C. to about 100° C. for about 30 seconds and a second temperature of about 101° C. to about 103° C. for about 4.5 minutes.

### Detection of Nucleic Acids

The presence of amplified target sequences or amplicons may be detected by any of a variety of well-known methods. For example, in some embodiments electrophoresis may be used (e.g., gel electrophoresis or capillary electrophoresis). Amplicons may also be subjected to differential methods of detection, for example, methods that involve the selective detection of variant sequences (e.g., detection of single nucleotide polymorphisms or SNPs using sequence specific probes). In some embodiments, amplicons are detected by real-time PCR.

Increased endpoint fluorescence above baseline noise levels enable result calling by real-time PCR, though a significant increase in fluorescence is important for accurate quantification. Inhibition of PCR due to inhibitors present in a sample leads to lower fluorescence and inaccurate threshold (Ct) determination when using quantitative PCR threshold analysis methods (Guescini et al. BMC Bioinformatics (2008) 9:326).

Real-time PCR or end-point PCR may be performed using probes in combination with a suitable amplification/analyzer such as the Spartan DX-12 desktop DNA analyzer, or the Spartan Cube which are low-throughput PCR systems with fluorescent detection capabilities. Briefly, probes specific for the amplified target sequence (e.g., molecular beacons, TaqMan probes) are included in the PCR amplification reaction. For example, molecular beacons contain a loop region complementary to the target sequence of interest and two self-complementary stem sequences at the 5′ and 3′ end. This configuration enables molecular beacon probes to form hairpin structures in the absence of a target complementary to the loop. A reporter dye is positioned at the 5′ end and a quencher dye at the 3′ end. When the probes are in the hairpin configuration, the fluorophore and quencher are positioned in close proximity and contact quenching occurs. During PCR, the fluorescently labeled probes hybridize to their respective target sequences, the hairpin structure is lost, resulting in separation of the fluorophore and quencher and generation of a fluorescent signal. In another example, TaqMan probes contain a reporter dye at the 5′ end and a quencher dye at the 3′ end. During PCR, the fluorescent labeled TaqMan probes hybridize to their respective target sequences; the 5′ nuclease activity of the DNA polymerase (e.g., Taq polymerase) cleaves the reporter dye from the probe and a fluorescent signal is generated. When probes that hybridize to different target sequences are used, these are typically conjugated with a different fluorescent reporter dye. In this way, more than one target sequence may be assayed for in the same reaction vessel. The increase in fluorescence signal is detected only if the target sequence is complementary to the probe and is amplified during PCR. A mismatch between probe and target sequences greatly reduces the efficiency of probe hybridization and cleavage.

Nucleic acids are routinely analyzed for clinical diagnosis, prognosis and treatment of diseases and conditions such as heritable genetic disorders, infections due to pathogens, and cancer. Generally the sample type analyzed is a biological sample such as a cell sample, body fluid sample, or swab sample. Nucleic acid analysis is also performed for detection of contaminating pathogens in environmental samples such as industrial water samples. Commonly used analysis methods include a step of extracting or purifying the nucleic acid from the sample prior to amplification. However, this step takes additional time, often requires use of expensive and/or special reagents and can result in loss or degradation of the nucleic acid. Therefore, methods that do not require extraction or purification of the nucleic acid prior to performing amplification (e.g., directly contacting the sample with the nucleic acid amplification reagent) are advantageous. Challenges to overcome when using methods that directly analyze a sample include the presence of PCR inhibitors in the sample and/or low concentration of nucleic acid. Real-time PCR-based methods have been successfully applied to Legionella monitoring of hot sanitary water (which can be described as “clean water”).

However, PCR-based testing and monitoring of “dirty water” samples, that may also comprise various organic and inorganic contaminants (e.g.. from industrial cooling tower systems, untreated freshwater), for microorganisms has proven challenging. The contaminants found in these water sources are often inhibitors of nucleic acid polymerases. Attempts to extract or purify the nucleic acid from the samples prior to amplification have had mixed success. In some instances, the nucleic acid is degraded or otherwise lost from the sample, or the inhibitors are inefficiently removed.

The effects of PCR inhibitors co-extracted with DNA from industrial cooling tower water systems can be mitigated by further dilution of the sample. However, this may result in a decreased sensitivity of the method, especially when the abundance of Legionella in the water is low, leading to false-negative results (Baudart et al., J App Micro (2015) 118(5):1238-1249).

Purification or extraction of DNA from the sample may also mitigate the effects of PCR inhibitors. Diaz-Flores et al. performed quantitative PCR on 65 water samples collected from cooling towers, sanitary water, nebulizer and spa matrices (BMC Microbiol (2015) 15:91). Prior to PCR the samples were treated with a lysis buffer, vortexed, incubated at 95° C. and vortexed again to collect the DNA. However, even with this level of purification, 8 of 65 samples (12.3%) demonstrated partial or complete inhibition of PCR.

Even when DNA is extracted from environmental water samples, there is still an appreciable PCR inhibition rate. For example, PCR inhibition was observed in 2.7% of DNA samples extracted from water collected from 37 cooling towers following concentration and filtration of the water and purification of the DNA using a High Pure PCR template preparation kit (Roche Diagnostics) (Joly et al., Appl. Environ. Microbiol. 7 (2006) 2(4): 2801-2808). In another study, PCR inhibition was observed in 5% of DNA samples extracted from water collected from cooling water towers for detection of Legionella (Ng et al., Lett. Appl. Microbiol. (1997) 24(3):214-16).

Legionella may also be quantified by culture methods, however contamination may not be detected, or underestimated, in some samples. The CDC conducted proficiency testing of 20 culture laboratories and found that Legionella concentrations in water samples were underestimated by an average of 1.25 logs or 17-fold (Lucas et al., Water Res. (2011) 45:4428-4436). Also, culture testing incorrectly reported water samples as negative for Legionella an average of 11.5% of the time when in fact they were positive. Furthermore, standard procedures for recovery of Legionella, including shipping. filtration, and heat/acid enrichment, are known to lead to a significant loss of cell culturability (Boulanger and Edelstein, J. Appl. Microbiol. (1995) 114:1725-1733; McCoy et al. Water Res. (2012) 46:3497-3506: Roberts et al., Appl. Environ. Microbiol. (1987) 53:2704-2707). Furthermore, culture testing is logistically disadvantageous as it requires shipment of samples to a central laboratory and 10-14 days for Legionella growth.

### EXEMPLIFICATION

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1

The present example illustrates a nucleic acid amplification reaction in accordance with some embodiments of the present disclosure.

Six water samples were collected from cooling towers. 110 mL of each of sample were concentrated using a 0.45 μm polyethersulfone (PES) 33-mm filter disk and a syringe pump. The filter was washed with 20-30 mL of distilled water and pulsed back and forth 10 times with 100 μL of distilled water. A final eluent was extracted in two 100-μL fractions of the concentrated sample. The two 100-μL fractions were pooled to create a 200-μL eluate.

The eluate was diluted 1:1 with a mastermix with concentrations of reaction components as shown in Table 1.

TABLE 1
Concentrations of the reaction components of the mastermix.

| Reaction components | Final concentration | |
|---|---|---|
| 5X Colourless GoTaq ® Reaction Buffer | 1X | |
| (Promega, Cat. No. M792B) | | |
| dNTPs (Enzymatics, Cat. No. N2050L) | 0.3 | mM |
| Magnesium chloride (Sigma, Cat. No. M1028) | 0.5 | mM |
| GoTaq ® MDx Hot Start Polymerase | 8 | Units |
| (Promega, Cat. No. D6001) | | |
| Lpn FOR primer (“Target forward primer”) | 1.5 | μM |
| Lpn REV primer (“Target reverse primer”) | 1.5 | μM |
| Lpn probe (“Target probe”) | 1.95 | μM |
| Legionella pneumophila genomic DNA | 500 Genomic | |
| (ATCC, Cat. No. 33152D-5) | Units | |
| S3_WT_MBP-1 (control primer) | 5 | μM |
| S3_WT_probe (control probe) | 0.74 | μM |

In some embodiments, a Magnesium chloride concentration of about 1.7 mM may be used.

Table 2 shows the sequences of the above described primers and probes:

TABLE 2
Primer and probe sequences.

| Name | Sequence |
|---|---|
| Lpn FOR primer | 5′-TTGTCTTATAGCATTGGTGCCG-3′ |
| Lpn REV primer | 5′-CCAATTGAGCGCCACTCATAG-3′ |
| Lpn probe | 5′-CAL_610- |
| | CAATTGAGCGCCACTCATAG-BHQ-2-3′ |
| S3_WT_MBP-1 | 5′-ATCCAGGG-3′ |
| S3_WT_probe | 5′-FAM_ccgacgTGTAAGCACCCCCTGGATCcgt |
| | cgg_BHQ-1-3′ |

The mastemnix and eluate mixtures were aliquoted into Spartan Cube® reaction cartridges (Spartan Bioscience, Part No. 01004977) and inserted into a Spartan Cube® thermal cycling device (Spartan Bioscience, Part No. 01014187). Table 3 shows the thermal cycling programs performed:

TABLE 3
Cycling activity.

| Temperature | Time |
|---|---|
| (° C.) | (sec) |
| 102.5 | 30 |
| 99 | 270 |
| 47 | 0 |
| 62 | 15 |

Cycle 49X

| | |
|---|---|
| 102.5 | 30 |
| 47 | 0 |
| 62 | 15 |

A control sample was also tested. The control sample consisted of tap water.

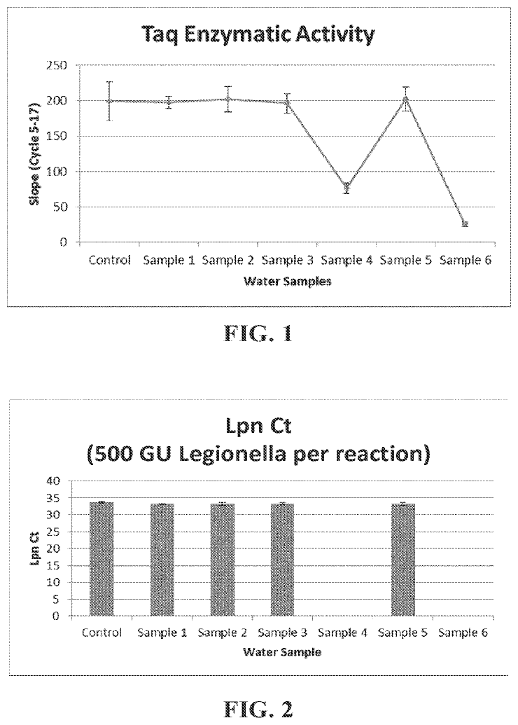

FIG. 1 shows Taq enzymatic activity in six samples and a positive control using six replicates per sample. Slopes of amplification plots (fluorescence against number of cycles) were calculated. Samples 1-3, and 5, and the control, exhibited similar slopes indicating comparable amplification behavior. Samples 4 and 6 exhibit a decreased slope value. Such a value indicates that the sample was inhibitory to the function of Taq polymerase.

FIG. 2 shows average cycle threshold (Ct) values from Legionella (Lpn) qPCR cycling in six samples and a positive control using six replicates per sample. Similar to the results from the Taq enzymatic activity cycling, Samples 4 and 6 did not produce a result. This indicates complete inhibition of qPCR.

Overall, results from the Taq enzymatic activity cycling correlated strongly with inhibition of cycle threshold (Ct) values with qPCR.

### Example 2

The present example illustrates a nucleic acid amplification reaction in accordance with some embodiments of the present disclosure. Results indicate that high concentration of competitive internal amplification control (IAC) may impair detection of target polynucleotides.

PCR primers and probes were designed against conserved regions in the Legionella pneumophila genome. In addition, a competitive internal amplification control (TAC) was created that consisted of a linearized recombinant plasmid with an insert of a 68-base pair human RNaseP sequence flanked by the Legionella forward and reverse primer sequences. Table 4 shows the sequences of the above described primers and probes for the example Legionella assay and competitive IAC.

TABLE 4
Primer and probe sequences.

| DNA component | Sequence (5′-3′) |
|---|---|
| Legionella forward primer | TTGTCTTATAGCATTGGTGCCG |
| Legionella reverse primer | CCAATTGAGCGCCACTCATAG |
| Legionella probe | CalFluor610_CGGAAGCAATGG |
| | CTAAAGGCATGCA_BHQ-2 |
| RNaseP probe | Fam_TTCTGACCTGAAGGCTCTGC |
| | GCG_BHQ-1 |
| IAC recombinant plasmid | TTGTCTTATAGCATTGGTGCCGAT |
| insert | TTGGGGAAGAATTTTAAAAATCAA |
| | GGCATAGATGTTAATCTTCTGACC |
| | TGAAGGCTCTGCGCGAGACGCTAT |
| | GAGTGGCGCTCAATTGG |

A mastermix was assembled and dispensed into a 96-well PCR plate per Table 5 below. Legionella genomic DNA was added to each well (25, 250, 1000, or 2500 genomic units (GU)). IAC plasmid DNA was added to each well (0, 25, 250, 2500 or 25000 copies) so that all ratios of Legionella GU:IAC copy number were dispensed.

TABLE 5
Final concentrations of the reaction components
used for the Legionella mastermix.

| | | Final | |
|---|---|---|---|
| Component | Supplier | Concentration | |
| 5x Colourless GoTaq | Promega M792B | 1x | |
| Reaction Buffer | | | |
| dNTPs | Enzymatics N2050L | 0.2 | mM |
| MgCl2 | Promega A3511 | 2.5 | mM |
| GoTaq MDx Hot Start | Promega D6001 | 2 | units |
| Polymerase | | | |
| Legionella forward | IDT custom product | 1.3 | μM |
| primer | | | |
| Legionella reverse | IDT custom product | 1.3 | μM |
| primer | | | |
| Legionella probe | Biosearch custom product | 1 | μM |
| RNaseP probe | Biosearch custom product | 1 | μM |
| BSA | Sigma 5470 | 40 | μg/μL |

| | | |
|---|---|---|
| Legionella pneumophila | ATCC 33152D | Range |
| genomic DNA | | of inputs |
| Competitive IAC | BioBasic custom order | Range |
| recombinant plasmid | | of inputs |

PCR reactions were run on an Applied Biosystems (ABI) QuantStudio3 real-time PCR system. The thermal cycling program was: 95° C. x 5 minutes; (95° C. x 5 sec, 62° C. x 12 sec)×45 cycles.

FIG. 3 shows the effect of IAC copy number on Legionella crossing thresholds (Ct). PCR reactions were performed with different input ratios of target (Legionella) to internal control (IAC). The smallest ratios of Legionella to IAC resulted in no detection of target (i.e., Ct=50).

The results indicated that as the ratio of Legionella GU:IAC copy number decreased. sensitivity diminished for Legionella detection. For example, at a ratio of 25 GU Legionella:2500 copies IAC, no Legionella was detectable in the PCR reaction (i.e., crossing threshold (Ct)=50) in this experiment.

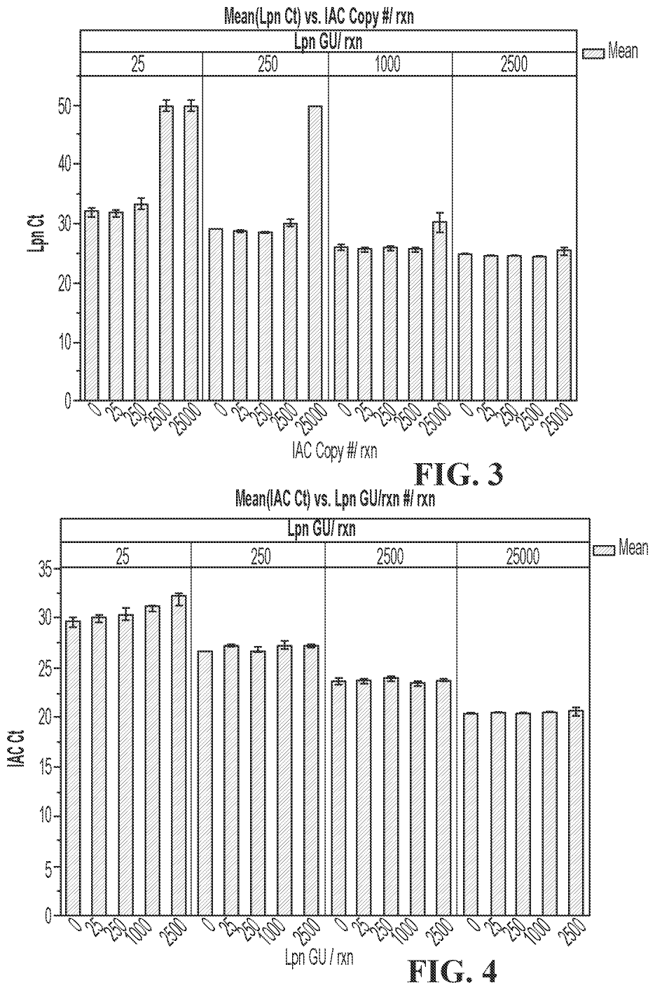

FIG. 4 shows the effect of Legionella GU on IAC Ct. PCR reactions were performed with different input ratios of internal control (IAC) to target (Legionella). The smallest ratios of IAC to Legionella resulted in delayed TAC Ct. The results also indicated that as the ratio of IAC copy number:Legionella GU decreased, sensitivity diminished for detection of the IAC. For example, at a ratio of 25 copies IAC:2500 copies Legionella, the Ct was delayed to 32, compared to a Ct of 29 when no Legionella was present in this experiment.

### Example 3

The present example illustrates a nucleic acid amplification reaction in accordance with some embodiments of the present disclosure. Results indicate that high concentration of non-competitive internal amplification control (IAC) impairs detection of target polynucleotides.

PCR primers and probes were designed against conserved regions in a Legionella pneumophila genome. In addition, a non-competitive IAC was created that consisted of a linearized recombinant plasmid with an insert of a 68-base pair human RnaseP sequence flanked by RnaseP forward and reverse primer sequences. Table 6 shows the sequences of the above described primers and probes for the example Legionella assay and non-competitive JAC.

TABLE 6
Primer and probe sequences.

| DNA component | Sequence (5′-3′) |
|---|---|
| Legionella forward primer | TTGTCTTATAGCATTGGTGCCG |
| Legionella reverse primer | CCAATTGAGCGCCACTCATAG |
| Legionella probe | CalFluor610_CGGAAGCAAT |
| | GGCTAAAGGCATGCA_BHQ-2 |
| RnaseP forward primer | GGACGGTCATGGGACTTCAG |
| RnaseP reverse primer | AAGGTGAGCGGCTGTCTCC |
| RnaseP probe | Fam_TTCTGACCTGAAGGCTCT |
| | GCGCG_BHQ-1 |
| Non-competitive IAC | GGACGGTCATGGGACTTCAGCA |
| oligonucleotide | TGGCGGTGTTTGCAGATTTGGA |
| | CCTGCGAGCGGGATCTATCACA |
| | TTCTGACCTGAAGGCTCTGCGC |
| | GGACTTGTGGAGACAGCCGCTC |
| | ACCTT |

A master mix was assembled and dispensed into Spartan Cube* PCR reaction tubes (Spartan Bioscience Ontario, Canada). Final concentrations of the reaction components used for the Legionella mastermix are shown in Table 7. 100 Genomic Units (GU) of Legionella genomic DNA were added to each reaction and 600 copies of non-competitive IAC DNA were added to half of the samples.

TABLE 7
Final concentrations of the reaction components
used for the Legionella mastermix.

| | | Final | |
|---|---|---|---|
| Component | Supplier | Concentration | |
| 5x Colourless GoTaq | Promega M792B | 1x | |
| Reaction Buffer | | | |
| dNTPs | Enzymatics N2050L | 0.3 | mM |
| MgCl2 | Promega A3511 | 1.7 | mM |
| GoTaq MDx Hot Start | Promega D4101 | 8 | units |
| Polymerase | | | |
| Legionella forward | IDT custom product | 1.6 | μM |
| primer | | | |
| Legionella reverse | IDT custom product | 1.6 | μM |
| primer | | | |
| Legionella probe | Biosearch custom product | 2 | μM |
| RnaseP forward primer | IDT custom product | 0.8 | μM |
| RnaseP reverse primer | IDT custom product | 0.8 | μM |
| RnaseP probe | Biosearch custom product | 0.8 | μM |
| Legionella pneumophila | ATCC 33152D | 100 | GU |
| genomic DNA | | | |
| IAC non-competitive | IDT custom product | 600 | copies |
| oligonucleotide | | | |

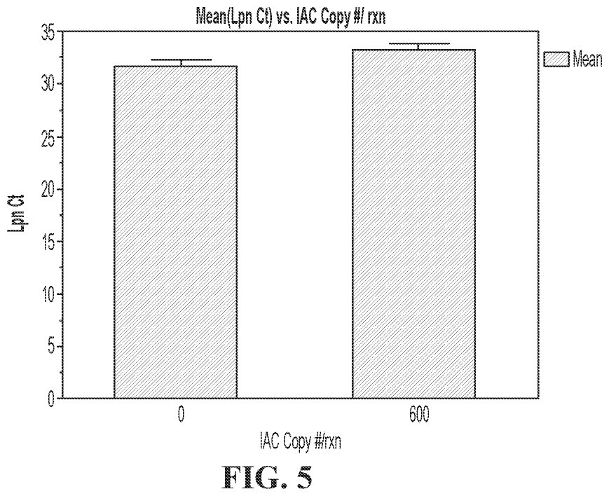

FIG. 5 shows the effect of multiplexing a non-competitive IAC reaction on Legionella crossing threshold (Ct). PCR reactions for 100 GU of Legionella DNA were performed with (right) and without (left) a non-competitive IAC reaction. Results showed that the crossing threshold (Ct) for 100 GU of Legionella in a singleplexed reaction was 32. When a non-competitive IAC reaction is multiplexed with the Legionella reaction, the Ct is delayed to 33.5. Inclusion of the non-competitive IAC delayed the Legionella Ct.

### Other Embodiments and Equivalents

While the present disclosures have been described in conjunction with various embodiments, and examples. it is not intended that they be limited to such embodiments, or examples. On the contrary, the disclosures encompass various alternatives, modifications, and equivalents, as will be appreciated by those of skill in the art. Accordingly, the descriptions. methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments, that are functional and/or equivalents of the specific embodiments, and features that have been described and illustrated. Accordingly, for example, methods and diagrams of should not be read as limited to a particular described order or arrangement of steps or elements unless explicitly stated or clearly required from context (e.g., otherwise inoperable). Moreover, the features of the particular examples and embodiments, may be used in any combination. The present disclosure therefore includes variations from the various examples and embodiments, described herein, as will be apparent to one of skill in the art.

1.-13. (canceled)

14. An internal amplification control for a nucleic acid amplification reaction, comprising:
a single oligonucleotide primer; and
a nucleic acid template,
wherein when present with a target nucleic acid sample and contacted with a nucleic acid amplification reagent in a reaction vessel, the nucleic acid template linearly amplifies and the target nucleic acid sample exponentially or linearly amplifies, and
wherein when the nucleic acid template is present at a higher concentration than the target nucleic acid sample, amplification of the nucleic acid template does not consume nucleic acid amplification reagents at a faster rate than amplification of the target nucleic acid sample.

15. The internal amplification control of claim 14, wherein when an internal amplification control is amplified its cycle threshold, slope or end point fluorescence is determined.

16. The internal amplification control of claim 14, comprising a reference sample.

17. The internal amplification control of claim 14, wherein a sequence of the nucleic acid template is or comprises the single oligonucleotide primer or a sequence complementary to the single oligonucleotide primer.

18. The internal amplification control of claim 14, comprising a probe.

19. The internal amplification control of claim 14, wherein when the single oligonucleotide primer binds to a nucleic acid template, the single oligonucleotide primer is extended by a polymerase.

20. The internal amplification control of claim 14, wherein when the single oligonucleotide primer is extended by the polymerase, a probe activates.

21. The internal amplification control of claim 14, wherein the nucleic acid template is a plasmid that has more than one complementary sequence to the single oligonucleotide primer and a hydrolysis probe.

22. The internal amplification control of claim 14, wherein activation of a probe produces a fluorescence signal.

23. The internal amplification control of claim 14, wherein the nucleic acid amplification reagent comprises a DNA polymerase at a concentration of at least about 8.0 U/reaction and a target specific primer concentration of at least about 1.5 p M, and a single oligonucleotide primer concentration of at least about 5.0 p M.

24. The internal amplification control of claim 14, wherein the nucleic acid template and the target nucleic acid sample are present in the reaction vessel at a relative quantity, wherein the relative quantity is a ratio of nucleic acid template copy number to nucleic acid sample genomic units, wherein said ratio is at least about 0.001, about 0.005, about 0.01, about 0.05, about 0.1, about 0.5, about 1.0, about 1.5; about 2.0, about 2.5, about 5.0, about 10.0, about 15.0, about 20.0, about 50.0, about 100.0, about 500.0, or about 1000.0.
